(19) United States
(12) Patent Application Publication
(10) Pub. No.: US 2009/0048569 A1
(43) Pub. Date: Feb. 19, 2009

(54) DISPOSABLE FEMALE URINARY AID

(76) Inventor: MARJAN SALEHI, Paradise Valley, AZ (US)

Correspondence Address: HOLME ROBERTS & OWEN LLP, 16427 N. SCOTTSDALE RD., SUITE 300, SCOTTSDALE, AZ, 85254, US

(21) Appl. No.: 12/193,675

(22) Filed: Aug. 18, 2008

Related U.S. Application Data
(60) Provisional application No. 60/956,379, filed on Aug. 16, 2007.

(51) Int. Cl. A61F 5/44

(52) U.S. Cl. 604/347

(57) ABSTRACT

The present disclosure provides for a device to facilitate urination by women in an upright position. The present device includes an opening that fits at least over the female labia with the rim contoured to surround the female genital region. A semi-rigid body comprised of a tri-layered latex-paper-wax funnel depends from the latex-rolled rim and slopes downward from the circumference of the rim extending into a latex-only tubular portion that collapses into the funnel for packaging and then be pulled back into the funnel via a drawstring for easy disposal.

### CLAIM TO DOMESTIC PRIORITY

This application claims priority to U.S. Provisional Patent Application by Marjan Salehi entitled “Detachable and Disposable Female Urinary Device,” Ser. No. 60/956,379, filed Aug. 16, 2007.

### FIELD OF THE INVENTION

This invention relates generally to the field of female urinary devices and more specifically to a latex-based female urinary cup designed to assist females in urinating in a standing position.

### BACKGROUND OF THE INVENTION

Women's public restrooms can be unclean and unsanitary in nature. Further, even in relatively clean public restrooms, toilet seats may be wet or dirty. Due to the female anatomy, urine flow is controlled primarily through gravity and, unlike in males, the flow cannot be directed. This leads to difficulty controlling urine flow when women are faced with situations where toilet seats are either unavailable or undesirable for use. For example, when hiking or backpacking, a toilet seat is usually unavailable. A woman must lower her clothing, exposing much of her lower body to the outdoor elements, squat while holding the clothing, and simply hope that when the urine flows, it does not hit her clothing or shoes. Even after taking all precautions, there is no guarantee that clothing or shoes will not get wet with urine since the direction of the flow cannot be controlled.

This is also the case with young girls who are unaccustomed to going to the bathroom without a toilet seat. Emergency stops along the road to go to the bathroom can be very difficult for women and girls (and their parents or caretakers) given the uncertainty in urine flow direction.

Women also are faced with situations when an available toilet seat is undesirable to sit on, such as in public restrooms, unisex restrooms, outdoor portable toilets, airplanes, tour busses, trains, and so on. These toilets are often dirty or otherwise in an unsanitary condition and women do not want to put their legs in contact with the toilet seat. To avoid sitting on an unsanitary toilet seat, women must pull down clothing and squat, holding themselves up over the toilet without touching any part of their legs to the toilet seat or rim.

While maintaining this awkward and difficult squatting position, women must release their bladder muscles and hope that the urine stream will reach the toilet bowl without hitting the toilet seat, toilet rim, their legs, clothes and/or the bathroom floor. It is difficult for women to partially squat and release urine at the same time. The leg muscles are unaccustomed to holding a woman's body in place while squatting in a backwards direction with the width of the leg separation limited by pulled down clothing such as pants and underwear. It is a balancing act and can be very stressful if urine flows onto the woman's body or clothes.

In addition, while toilet seats are common in the United States, many cultures in other countries do not customarily build bathrooms designed for women and/or do not have toilet seats installed in bathrooms. Most women have encountered bathrooms while traveling outside of the United States that are designed for the male anatomy in the sense that the urine is to be directed towards a target—whether the target is a urinal or a hole in the floor which a woman must straddle. Without the natural ability to direct urine flow, these types of bathroom facilities present similar problems for women—they are forced to remove or pull down clothing, straddle a target that is usually placed in front of them, and release their bladder muscles without knowing where the flow of urine will be directed other than in a generally downward direction, controlled by gravity alone.

There also are instances when a woman may not be physically comfortable, or physically able, to sit on a toilet seat, such as following surgery. Examples include hip injury, post hip surgery, arthritis, or any condition causing pain or discomfort to a woman if she has to bend into a squatting or sitting position. It is also awkward and difficult for women to provide urine samples in a medical setting since the woman must hold a cup underneath her vulva and over the toilet bowl hoping that the urine stream will find its way into the cup and not elsewhere.

Situations where toilet seats are not available or in an undesirable/unsanitary condition are often accompanied by a confounding problem of a lack of toilet paper with which to wipe the vulva when finished urinating. Given the female anatomy, women are unable to tap the last drops of urine from their vulva and must instead use something to wipe the residual moisture away. If toilet paper is unavailable, a woman must let the urine be absorbed into the underwear leaving an uncomfortable sensation of moisture.

Existing devices constructed of paper can be folded flat for storage by means of two opposed creases that define the tapered vertical front and back profiles of the funnel. To use them, pressure is applied to the creases to flex open the funnel. However, these devices must be held by a user around its exterior and pressure must be carefully maintained across the creases to flex and keep the device open during use. The urine flow must be carefully directed into the funnel opening, since the funnel absorbs urine, which can cause it to collapse.

Further, the changes in weight due to variable urine flow requires a very deft amount of flex pressure on the creases to keep the funnel uniformly open. Further, if it leaks and/or overflows, the user's hand will get wet with urine. The device is also designed to absorb the urine flow, leaving a wet funnel when finished.

Accordingly, there is an unmet need for a disposable female urination device to direct urine which is sufficiently waterproof and firm to serve the dual purposes of directing the urine flow while protecting the user's hand from contact with urine, on the one hand, and is simple and comfortable to use by an adult woman, girl, or the caretaker of a girl in a variety of conditions and situations where toilet seats are unavailable or unsuitable. Further, the device should possess a mechanism that makes disposal of the device simple and capable of preventing residual urine from dripping or otherwise escaping from the device.

Therefore, a need exists for a new, more effective female urinary device that can be easily disposed of following use.

### BRIEF DESCRIPTION OF THE DRAWINGS

FIG. 1 is a side view of the device in an open configuration.

FIG. 2 is a top view of the device showing the layered construction of die upper funnel portion.

FIG. 3 is a bottom view of the discharge opening of the device.

FIG. 4 is a side view of the device in use.

FIG. 5 is a view of the device ready for disposal.

FIG. 6 is a front view of a packaged device, either before use or after the device has been replaced in the package for disposal.

FIG. 7 is a front view of the device adapted to use in a sitting or laying position, such as in hospitals.

FIG. 8 is a front view of the device with a urine storage balloon.

### DETAILED DESCRIPTION OF VARIOUS EMBODIMENTS

The present invention concerns a device to facilitate urination by women in an upright position. The present device includes a flexible or semi-rigid opening that fits at least over the female labia with the rim contoured to surround the female genital region. A flexible body depends from the rim and slopes downward from the circumference of the rim terminating at a tube. A bendable, elongated tube extends from the funnel portion and is inclined downward and outward. The tube is sufficiently flexible to enable bending for packaging and for directing urine flow.

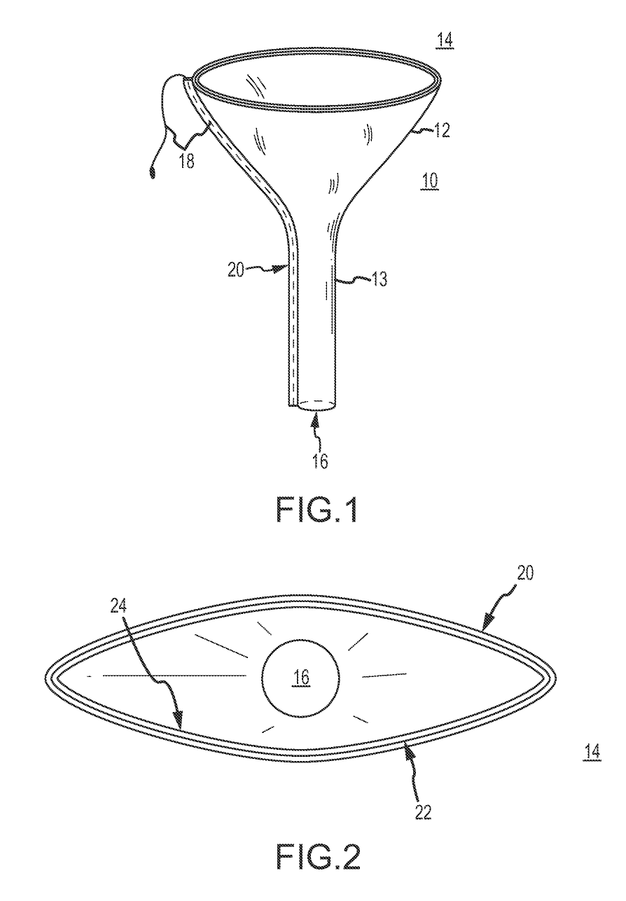

FIG. 1 is a side view of the device in an open configuration. As shown in FIG. 1, the disclosed female urination device 10 comprises a generally funnel-shaped body 12 made primarily of disposable latex material or any other material known in the art that is suitable to the disclosed construction and purpose. The body 12 has a continuous funnel-shaped wall and an elongated discharge tube 13 extending from the bottom of the funnel-shaped body terminating in discharge opening 16.

FIG. 2 is a top view of the device showing the layered construction of the upper funnel portion. As shown in FIG. 2, the opening and funnel shaped portion 14 of the body 12 of the device is constructed in a layered manner with a latex 20 exterior layer, a stiff paper 22 middle layer and a wax coating 24 interior layer. In one embodiment, the funnel is constructed from a relatively firm paper or cardboard 22 that is pre-coated with wax 24 on the interior. The wax-coated paper funnel is then dipped in latex using a mold to create the elongated tube portion 13, creating the body of device 10. Thus, the tubular portion 13 of the body is comprised only of the latex material. The more flexible, latex-only tubular portion 13 allows for easier packaging and disposal as it is capable of folding in under the circumference of the opening of the funnel. Thus, the device folds flat for packaging, carry storage and disposal.

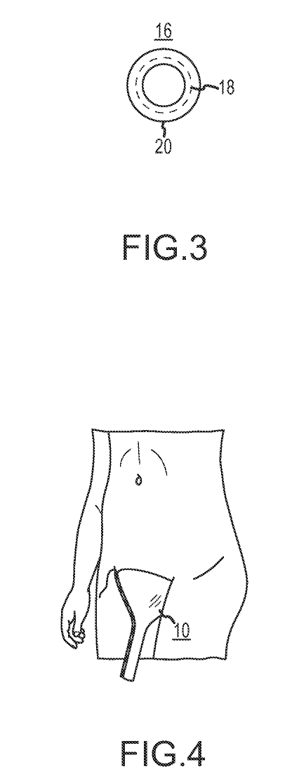

FIG. 3 is a bottom view of the discharge opening 16 of the device. As shown in FIG. 1 and FIG. 3, the device 10 is equipped with a drawstring 18. Drawstring 18 is encased in tubular latex housing 20 that runs the circumference of discharge opening 16 and up the side of body 12 terminating in an opening at the top portion of funnel 14. Drawstring 18 completely surrounds discharge opening 16 and runs up through the housing and out the opening in the top portion of funnel 14. A large enough portion of drawstring 18 extends from the top opening in order to allow a user to pull drawstring 18 pulling the tubular portion 13 of the device 10 into the funnel portion 14 for disposal.

FIG. 4 is a side view of the device in use. In order to use the device, the user opens the package 30 at package opening 32, shown as FIG. 6, and removes the device 10. Upon removal, the device 10 appears as shown in FIG. 2. The user allows the folded portion, comprised of the bottom of funnel 14 and tubular portion 13, which has been tucked under the opening portion of the funnel 14 for packaging, to fall in the direction the urine should flow (e.g., into the toilet).

The user then grasps the opening circumference of the device 10 and flexes it as needed to secure the proper fit over the labia, as shown in FIG. 4. The device 10 may be placed with the left or right hand depending on the user's preference. Alternatively the rim or circumference of the device may have a spring-like mechanism formed by metal inserts or other material allowing the user to merely squeeze open the body of device without having to separate the walls manually. However, the funnel portion with a latex-paper-wax layered construction and latex-rolled rim of the funnel opening is firm enough to flex and hold device 10 in position.

The user may then hold the device in position between her legs and adjacent and/or touching her vulva while spreading her legs and/or crouching at the desired or most comfortable angle. The funnel opening is spread to the desired width depending on the shape of the user's body so as to comfortably fit within the space between the user's thighs immediately below the vulva. The funnel shape directs and controls the flow of urine towards the discharge opening at the bottom of the funnel and through the tube. The urine is projected out of the discharge opening and away from the user's body through the bottom tube opening shown in FIG. 3. The interior wax layer 24 is useful in continuing the flow of urine down the funnel to tubular portion 13 and preventing absorption of urine into paper layer 22. The latex-only construction of the tubular portion then directs the urine to its desired location. In an alternate embodiment, the wax layer may be replaced with any type of water resistant substance, such as a thin plastic film, a plasticized or resin-treated paper, or the like, so as to not be immediately permeable to urine passing through the device.

The device is capable of being used by one hand, freeing the other hand to help support the user of the device, if desired. The user may stand almost in an upright position when using the device to maximize balance and control. The device may optionally be held in position by another person, such as a parent or caretaker, when assisting a girl. Different sizes of the device may be made for girls, as opposed to women. The device is collapsible to a flat form, lightweight, easy to package and carry, such as in a pant pocket or purse, and is easy to store. It may be packaged separately or dispensed by means of a container with a slot opening such as in the manner of tissues.

In an alternate embodiment, the disclosed female urination device comprises a generally funnel-shaped body made of disposable, pliable, paper-based material or any other material known in the art that is suitable to the disclosed construction and purpose. The interior walls of the device are water resistant, e.g., thinly coated with wax, a thin plastic film, a plasticized or resin-treated paper, or the like, so as to not be immediately permeable to urine passing through the device. The outside walls of the device are absorbent to water/urine so that the user of the device may remove the device from her fingers when finished urinating and use the exterior of the device to wipe residual moisture from the vulva prior to disposal.

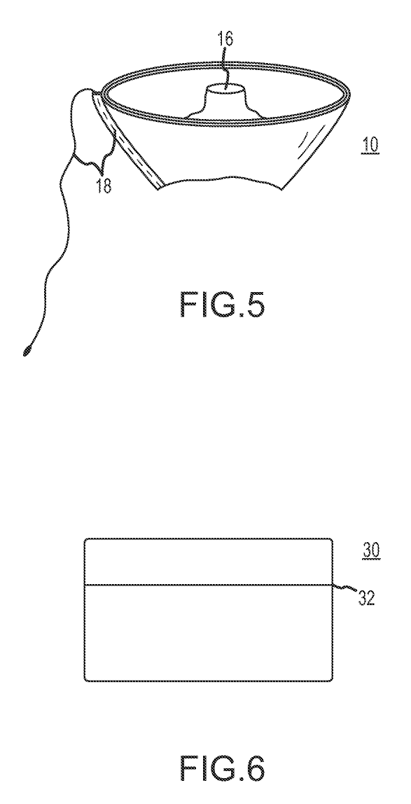

FIG. 5 is a view of the device ready for disposal. As shown in FIG. 5, after the device is used, the user pulls drawstring 18 causing the tubular portion 18 starting at discharge opening 16 to be pulled into funnel portion 14. This prevents any extraneous dripping of urine from discharge opening 16 and allows the user to compact the device for disposal without having residual urine escape from the device.

FIG. 6 is a front view of a packaged device, either before use or after the device has been replaced in the package for disposal. In one embodiment, the user replaces the compacted device into packaging 30 for disposal, and in some packaging, the user has the option of resealing the used device in the packaging by resealing opening 32. The used device in packaging 30 is then disposed of in any waste receptacle or feminine hygiene disposal container. This allows for clean and sanitary disposal of the used device.

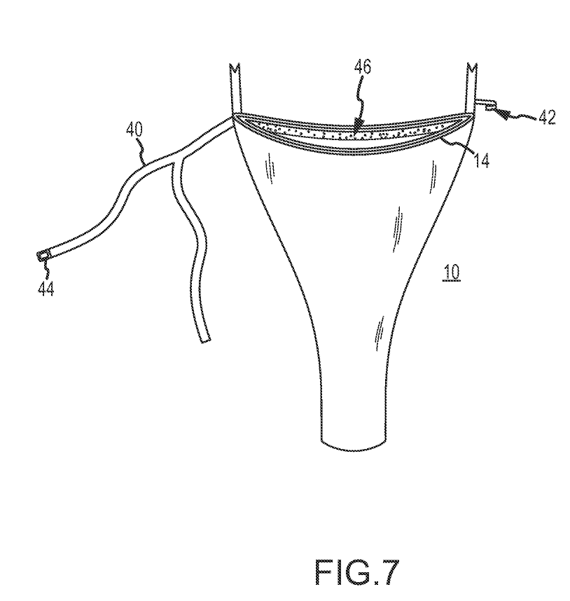

FIG. 7 illustrates an alternate embodiment of the device, namely, a version that can be tied around the thighs or hips to hold the device in place. The ties 40 can be elastic or some other comfortable, securing material, and can hook to the other side of the device with any type of hooking mechanism, such as hook 42 and eye 44 shown in FIG. 7. This embodiment has particular usefulness for those disabled or in hospital beds who cannot use a toilet and must urinate into a bed pan or other container. Because no hands are required, it is easy to use and prevents females from splattering urine in their beds or on their gowns. Additionally, an optional splash guard may be attached to the device to further prevent unwanted soiling of bedding or clothing. Further, this type of device may also comprise a non-stick adhesive 46 at the opening of funnel portion 14 (such as that used on suture bandages) to further secure the device around the labia.

Modifications within the scope of the appended claims are also envisioned. For example, the funnel portion can have a wide range of designs to provide the functionalities disclosed herein. There are also a variety of different paper-based materials that could be used and a variety of means for providing water resistance to the interior portion of the device and water absorbency to the exterior. Additionally, the packaged device may also contain a tissue or other type of wipe that can be used to wipe any remaining urine away from the labia or urethra.

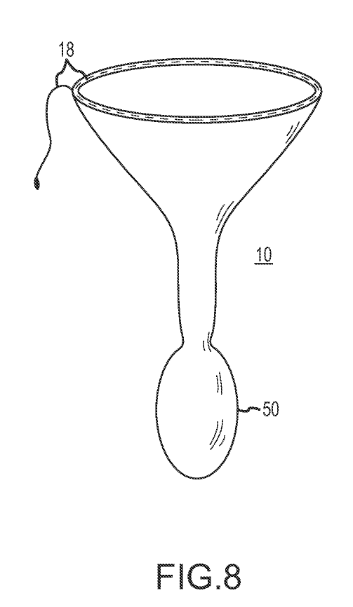

Further, the device may be used in conjunction with a disposal container or other type of mechanism for holding the urine for later disposal. FIG. 8 is one example of this and shows a front view of the device with the tubular portion terminating in a urine storage balloon 50 instead of having the discharge opening 16. As shown in FIG. 8, the tubular portion terminates in a latex balloon that is capable of holding urine and expanding as needed to hold the urine. This embodiment is particularly useful in cases where no acceptable place to urinate exists or in medical settings where the patient is unable to get out of bed, and especially in cases where the urine needs to be retained in order to be quantified or have laboratory analysis performed on it. In this embodiment, drawstring 18 runs the circumference of the opening of the funnel portion. Drawstring 18 is then pulled to close the top of the device closing the entire device and retaining the urine in balloon 50 without spillage out the top of the device.

It is clear that the disclosed device has wide applicability to the feminine hygiene industry, namely to controlling and directing urine flow when women are faced with situations where toilet seats are either unavailable or undesirable for use, and/or when a woman is not physically comfortable or physically able to sit on a toilet seat. The device may be used in a variety of situations, from traveling in locations that do not have adequate bathroom facilities for women, to outdoor use while hiking or backpacking, to emergency bathroom stops along the road when driving. The collapsible device is simple in design, easy to store, carry and use, and keeps the user's hands and body clean from urine. Thus, the inventive device has the clear potential of becoming adopted as the new standard device for assisting women in urinating when it is not possible, because of proximity, toilet seat conditions, physical pain and/or injury, to sit on a toilet seat.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment, and best mode of the invention known to the applicant at this time of filing the application, have been presented and is intended for the purposes of illustration and description. It is not intended to be exhaustive nor limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed is:

1. A water-resistant, collapsible, female urination aid comprising: a body including a funnel-shaped top portion with a receiving opening at the top of the funnel-shaped portion for receiving urine, an elongated tube extending from the bottom of the funnel-shaped portion with a discharge opening at the termination of the elongated tube for discharging urine from a user away from the user through the tube, wherein said funnel-shaped portion is comprised of self-supporting material having an external surface and an internal water-resistant surface to receive urine, and wherein the elongated tube is comprised of a flexible material that is collapsible into said funnel-shaped portion for packaging, and further including a drawstring extending from the discharge opening to the top of the funnel-shaped portion for pulling the elongated portion up into the funnel-shaped portion for disposal.

2. The device of claim 1, wherein the self-supporting material is comprised of wax-covered paper dipped in latex.

3. The device of claim 1, wherein the self-supporting material is comprised of an exterior layer, a middle layer and an interior layer.

4. The device of claim 3, wherein the exterior layer is comprised of latex.

5. The device of claim 3, wherein the middle layer is comprised of paper.

6. The device of claim 3, wherein the interior layer is comprised of wax.

7. The device of claim 1, wherein the device is individually packaged for use.

8. The device of claim 1, further including straps for attaching the device to a human, said straps being attachable around the thighs of the human.

9. The device of claim 1, wherein the device is packaged in an enclosed storage container having a slot opening to access at least one device stored inside said container.

10. The device of claim 1, further including a package for storage of the device prior to use and disposal of the device following use.

11. A water-resistant, collapsible, female urination aid comprising: a body including a funnel-shaped top portion with a receiving opening at the top of the funnel-shaped portion for receiving urine, an elongated tube extending from the bottom of the funnel-shaped portion with a discharge opening at the termination of the elongated tube for discharging urine from a user away from the user through the tube, wherein said funnel-shaped portion is comprised of a layered material to receive urine, said layered material having an external layer comprised of latex, a middle layer comprised of paper, and an interior layer comprised of wax, and wherein the elongated tube is comprised of a flexible material that is collapsible into said funnel-shaped portion for packaging, and further including a drawstring extending from the discharge opening to the top of the funnel-shaped portion for pulling the elongated portion up into the funnel-shaped portion for disposal.

12. The device of claim 11, further including straps for attaching the device to a human, said straps being attachable around the thighs of the human.

13. The device of claim 12, further including a non-stick adhesive for further securing the device to the human.

14. A water-resistant, collapsible, female urination aid comprising: a body including a funnel-shaped top portion with a receiving opening at the top of the funnel-shaped portion for receiving urine, an elongated tube extending from the bottom of the funnel-shaped portion with a balloon portion extending from the elongated tube for discharging urine from a user away from the user through the tube into the balloon, wherein said funnel-shaped portion is comprised of self-supporting material having an external surface and an internal water-resistant surface to receive urine, and wherein the elongated tube and balloon are comprised of a flexible material that permits expansion of the balloon as it fills with urine, and further including a drawstring extending around the circumference of top of the funnel-shaped portion for closing the device and preventing leakage of urine from the balloon portion.

15. The device of claim 14, wherein the self-supporting material is comprised of wax-covered paper dipped in latex.

16. The device of claim 14, wherein the self-supporting material is comprised of an exterior layer, a middle layer and an interior layer.

17. The device of claim 16, wherein the exterior layer is comprised of latex.

18. The device of claim 16, wherein the middle layer is comprised of paper.

19. The device of claim 16, wherein the interior layer is comprised of wax.

20. The device of claim 14, further including straps for attaching the device to a human, said straps being attachable around the thighs of the human.
